(19) United States
(12) Patent Application Publication
(10) Pub. No.: US 2019/0390156 A1
(43) Pub. Date: Dec. 26, 2019

(54) Open-cell Mycelium Foam and Method of Making Same

(71) Applicant: Ecovative Design LLC, Green Island, NY (US)

(72) Inventors: Eben Bayer, Troy, NY (US); Jacob Michael Winiski, Troy, NY (US); Matthew James Lucht, Cohoes, NY (US); Peter James Mueller, Poestenkill, NY (US); Gavin R. McIntyre, Troy, NY (US); Meghan A. O'Brien, Halfmoon, NY (US)

(73) Assignee: Ecovative Design LLC, Green Island, NY (US)

(21) Appl. No.: 16/444,354

(22) Filed: Jun. 18, 2019

Related U.S. Application Data
(60) Provisional application No. 62/688,427, filed on Jun. 22, 2018.

(51) Int. Cl. C12N 1/14; A01G 24/48; A01G 24/44; A01G 24/20; A01G 18/20

(52) U.S. Cl. CPC C12N 1/14; A01G 24/48; A01G 18/20; A01G 24/20; A01G 24/44

(57) ABSTRACT

The mycelial foam contains macroscopic void spaces that are formed by filler elements, such as agar beads, that are incorporated in the mycelial matrix during growth of the matrix and are removed from the matrix after growth in a non-destructive manner, such as by heating. The foam may be made of pure mycelium or may be a composite biomaterial.

This application claims the benefit of Provisional Patent Application 62/688,427, filed Jun. 22, 2018.

This invention relates to an open-cell mycelium foam and to a method of making the same. More particularly, this invention relates to a self-assembled fungal cellular matrix having a plurality of void spaces therein.

### BACKGROUND OF THE INVENTION

There are many industrial processes for producing a material which retains a percentage of the volume of the material as air space and this material is commonly referred to as foam. The creation of air spaces within these materials is often obtained by mixing fibrous, metallic or other base materials with additional materials, such as other solids or gasses that create void spaces within the base material.

U.S. Pat. No. 9,485,917 describes various techniques for using mycelium to bind together organic or inorganic materials into self-supporting molded shapes.

US 2015/0033620 describes a technique of producing a mycelium biopolymer.

Generally speaking, mycelium is a natural foam comprised of a matrix of hyphae that produce microscopic air spaces within the matrix.

It is an object of the invention to provide a light weigh mycelial foam that can be used for multiple applications.

It is another object of the invention to provide a relatively simple techniques for producing mycelial foams with macroscopic voids.

### BRIEF DESCRIPTION OF THE INVENTION

Briefly, the invention provides a mycelium foam, i. e. a self-assembled fungal cellular matrix, comprising a body of pure mycelium and a plurality of macroscopic void spaces with the body. In this respect, the term “macroscopic” means that the imposed void spaces are visible to the naked eye. This is in contrast to the natural cellular makeup of mycelium, which can be viewed as an open cell foam with void spaces created at the cellular level which are only viewable microscopically.

The term “macroscopic void spaces” used herein means void space sizes of from 0.04 inch (1 mm) to 3 inch. Typically, the void spaces constitute at least 10% of the body.

The invention utilizes the natural fungal process of hyphal growth to self-assemble the base material with no complex mixing of materials and binder.

In one embodiment, the invention creates a pure mycelial matrix with macroscopic void spaces sized in the millimeter and centimeter range (rather than microscopic) which forms an open cell foam using a technique similar to the lost-cast technique.

In another embodiment, the invention creates a composite matrix with macroscopic air spaces sized in the millimeter and centimeter range (rather than microscopic) and wherein the composite matrix, i.e. a composite biomaterial, includes non-nutrient discrete particles and mycelium.

The invention also provides a method for making a mycelium foam.

In one embodiment, the method comprises the steps of providing a tool defining a cavity therein; packing the cavity with a plurality of spaced apart filler elements; adding a fungus to the filler elements; and allowing the fungus to grow mycelium within the cavity to form a pure mycelial matrix and to allow the mycelium to individually encapsulate the filler elements with the matrix.

This embodiment also uses a step of removing the filler elements from the matrix to produce a fungal cellular matrix having a plurality of macroscopic void spaces therein.

In another embodiment, the method comprises the steps of providing a tool defining a cavity therein; packing the cavity with a substrate comprised of non-nutrient discrete particles and nutrient particles; adding a fungus to the substrate; allowing the fungus to grow mycelium within the substrate; thereafter adding a plurality of spaced apart filler elements to the substrate; and allowing the fungus to grow mycelium within the cavity to form a composite matrix of the non-nutrient discrete particles and mycelium and to allow the mycelium to individually encapsulate the filler elements with the composite matrix.

In each embodiment, final density of the foam is reduced by including filler elements in the matrices which occupy individually spaced apart volumes and which, by some non-destructive means, are replaced with a gas space in the final product.

These and other objects and advantages of the invention will become more apparent from the following detailed description taken in conjunction with the accompanying drawings wherein:

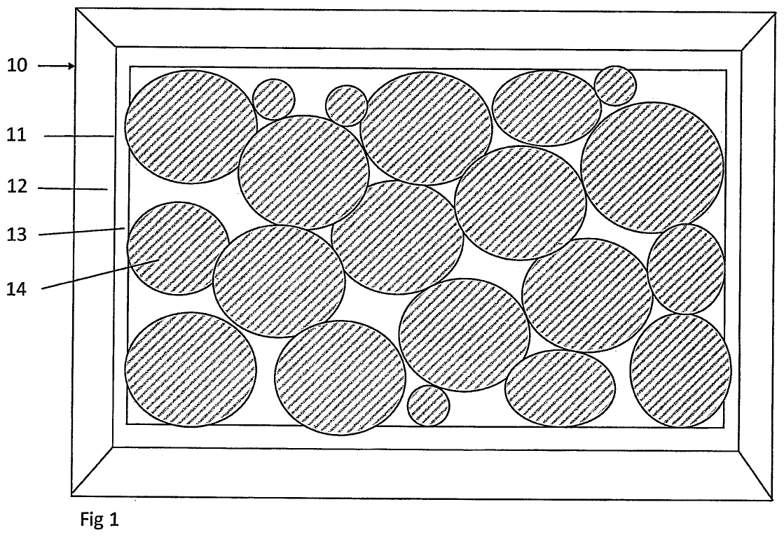

FIG. 1 illustrate a view of a tool of a cavity containing spaced apart filler elements in accordance with the invention;

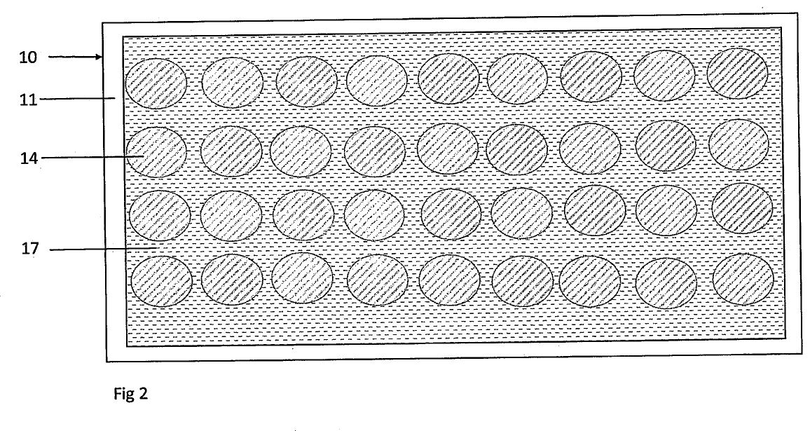

FIG. 2 illustrates a view of a tool of a cavity containing a composite substrate and spaced apart filler elements in accordance with the invention; and

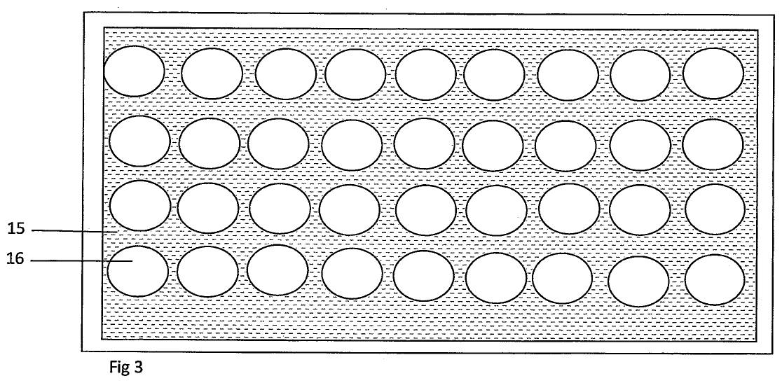

FIG. 3 illustrates a view of a composite matrix made in the tool of FIG. 2 having a plurality of macroscopic void spaces therein after removal of the filler elements in accordance with the invention.

The technique of the invention provides for the incorporation of components, such as filler elements, into a mycelial product where at least one component is designed in such a way that the component can be meaningfully removed from the final product by means other than physical removal and without damage to the product to create a self-assembled fungal cellular matrix comprised of a certain percentage of void space as affected by component removal.

The mycelium foam is comprised of at least one part self-assembled mycelial matrix, either with or without additional organic or inorganic substrate, and one part an incorporated removable element designed to provide a void space within the resultant matrix when removed through melting, desiccation, enzymatic or aqueous dissolution, or is in any way discluded from the matrix without disturbing the grown-structure.

The method of the invention utilizes the ability of fungal mycelium to grow into, through or around structures emplaced within a growth form as a self-binding matrix, as described in U.S. Pat. No. 9,485,917. This mycelial matrix can bind organic and inorganic substances, as well as bind to itself, while maintaining the shape imposed upon thereon by the growth form utilized.

Additionally, the matrix will grow around substances while either ingesting and incorporating nutrition from the substance to utilize for viability, or conversely, will grow around materials that are indigestible and maintain a binding network surrounding the material without any further interaction.

The invention utilizes materials generally, but not exclusively, of the indigestible type where the mycelial matrix is intended to grow around the removable component without ingesting the component.

At the conclusion of the prescribed mycelial growth period, the removable component is treated as required, leaving behind the mycelial matrix and any other inclusions not intended for removal, creating a self-assembled matrix with void spaces where the removed components once were. This, in effect, creates a natural, self-binding, foam-like product that is generally biodegradable and in most cases, will naturally and safely decompose when the action is desired.

Generally, the incorporation of objects that do not benefit or increase the vitality of an organism is not undertaken as the addition may be viewed as pointless or in some instances, harmful.

Referring to FIG. 1, one method of making a pure mycelium foam employed a step of providing a tool with a shallow cavity suitable for the shape of the foam to be made. As illustrated, the tool 10 had a rectangular shape and a cavity 11 with four sloped walls 12 and a flat bottom 13.

In accordance with the method, the cavity 11 of the tool 10 was packed with a plurality of spaced apart filler elements 14. In this example of the invention, the filler elements 14 were in the shape of beads and were composed of solidified agar. The bead diameter was customizable, as was agar nutrition. For example, potato dextrose agar, antibiotic-malt extract agar, or non-nutritious agar can be employed.

Next, a fungus (not shown) was added to the filler elements 14 in the tool 10 and allowed to grow mycelium within the cavity 11 to form a pure mycelial matrix and to allow the mycelium to individually encapsulate the filler elements 14 with the matrix.

The filler elements 14 to which the fungus is added may contain inherent levels of generally non-nutritive lingo-cellulosic material, or as is standard per US2015/0033620, additional, more easily accessible sources of nutrition, such as sugars, starches, and calcium, may be added to the filler elements 14 to further incite fungal colonization of the filler elements 14.

The fungal species can vary, but is generally selected to be a wood-rotting higher basidiomycete, in that the cellular structure of the organism most suitably offers the characteristics desired for product application and manipulation.

After encapsulation of the filler elements 14 with the matrix, the resultant mycelia matrix was removed from the tool 10 and treated to remove the filler elements 14 from the matrix to produce a fungal cellular matrix having a plurality of discrete macroscopic void spaces therein. In this respect, the void spaces were left behind by the agar beads after the agar beads were removed by being heated in a 180° F. oven for 24-hours.

The method as described provides a solely mycelial matrix wherein the final tissue product incudes discrete voids or pore spaces not otherwise able to be imparted on the matrix during growth.

As an alternative, the agar beads 14 may have an additive, such as a mineral solid, within each or at least some of the beads. In this embodiment, after removal of the agar, the additive would remain behind to occupy at least some of resulting otherwise void spaces.

Referring to FIG. 2, wherein like reference characters indicate like parts as above, one method of making a composite mycelium foam employed a tool 10, as above, that was packed with a substrate 17, as described in U.S. Pat. No. 9,485,917, comprised of non-nutrient discrete particles (not shown) and nutrient particles (not shown).

In addition, a plurality of spaced apart filler elements 14 were added to the substrate 17.

As illustrated in FIG. 2, the substrate 17 was a hemp regrind material supplied by Ecovative Design LC of Green Island, N.Y. and the filler elements 14 were agar beads interspersed with a grid pattern. The hemp regrind material in itself was a non-nutritive, partially digestible lingo-cellulose matter with a small added percentage of readily digestible nutrition, in this specific case, introduced as wheat flour, but can be substituted with other easily accessible nutrition as need warrants.

After, or before, applying the agar beads 14, a fungus (not shown) was added to the agar beads 14 in the tool 10 and allowed to grow mycelium within the cavity 11 to form a composite mycelial matrix of the non-nutrient discrete particles and mycelium and to allow the mycelium to individually encapsulate the agar beads 14 with the matrix.

During this growth period of time, the mycelium had a resistance to growing into the agar beads 14 and there was a lack of an inhibitory effect of the beads on proximal mycelial growth.

Referring to FIG. 3, after encapsulation of the agar beads 14 with the matrix, the composite mycelia matrix was removed from the tool 10 and treated to remove the agar beads 14 from the matrix to produce a fungal cellular composite matrix 15 having a plurality of macroscopic void spaces 16 therein. In this respect, the void spaces 16 were left behind by the agar beads 14 after the agar beads 14 were removed by being heated in a 180° F. oven for 24-hours.

The overall dimensions of the composite mycelia matrix 15 removed from the tool 10 were 1″×6″×12″, with void spaces around ¼ to ½″ in circumference.

The highly porous, and therefore lightweight, all-natural mycelial composites created in the manner described herein respond dually to the call for more environmentally friendly packaging and products as well as the call for packaging and products that are less costly (both monetarily and environmentally) to move from location to location vis-a\grave{ }-vis decreased gasoline usage and associated costs.

Further examples of the techniques of the invention follow.

### EXAMPLES

1.) Incorporation of agar beads of desired shape and size into an organic substrate with a mycelial matrix binder to create a hydrophobic, grown cellular matrix with foam-like properties derived from removal of the incorporated beads through melting.
   e.g.: The addition of 60 grams of agar beads approximately 2.5 mm in diameter to 70 dry grams of partially digestible, non-nutritive aspen chips approximately 1×1×1 mm in size, where air spaces are created after heat treatment at 180° F. for 24-hours and where the beads melt away. This results in a density decrease from 13 lb/ft³ to 10 lb/ft³.
2.) As above with removal of agar beads by desiccation, including convection, conduction and radiation.
3.) As above with removal of agar beads by aqueous dissolution.
4.) As above with removal of agar beads by enzymatic reaction.
5.) Incorporation of sodium polyacrylate beads round in shape and of variable size into an organic substrate with a mycelial matrix binder to create a hydrophobic, grown cellular matrix with foam-like properties derived from removal of incorporated beads through melting.
   e.g.: The addition of 250 grams of hydrated sodium polyacrylate beads approximately 1 mm in diameter into 70 dry grams of aspen chips approximately 1'1×1 mm in size, where air spaces are created after part treatment at 180° F. for 24-hours and where sodium polyacrylate beads melt away. This results in a density decrease from 13 lb/ft³ to 9 lb/ft³.
6.) As above with removal of sodium polyacrylate beads by desiccation, including convection, conduction and radiation
7.) Incorporation of beeswax round in shape and of variable size into an organic substrate with a mycelial matrix binder to create a hydrophobic, grown cellular matrix with foam-like properties derived from removal of incorporated beeswax through melting.
   e.g.: The addition of 60 grams of beeswax pellets approximately 1.0 mm in diameter to 70 dry grams of aspen chips approximately 1×1×1 mm in size, where air spaces of 1 mm were created after part treatment at 180° F. for 48-hours and where pellets melt away. This results in a density decrease from 13 lb/ft³ to 12 lb/ft³.
8.) Creation of foam-like, hydrophobic matrix through mycelial cellular growth as a binder of agar beads of desired shape and size that are removable through melting.
   e.g.: Inoculation with liquid or solid inoculum of a 15 mm petri dish holding thirty or more agar beads approximately 2.5 mm in diameter and where beads are removed through melting at 180° F. after at least 4-days of mycelial growth, leaving solely the mycelial matrix.
9.) As above with removal of agar beads by desiccation, including convection, conduction and radiation
10.) As above with removal of agar beads by aqueous dissolution
11.) As above with removal of agar beads by enzymatic reaction
12.) Creation of foam-like, hydrophobic matrix through mycelial cellular growth as a binder of sodium polyacrylate beads round in shape and of desired size that are removable through melting.
   e.g.: Inoculation with liquid or solid inoculum of a 15 mm petri dish holding ninety or more sodium polyacrylate beads approximately 1.0 mm in diameter and where beads are removed through melting at 180° F. for 24-hours after at least 7-days of mycelial growth, leaving solely the mycelial matrix.
13.) As above with removal of sodium polyacrylate beads by salt chemical reaction
14.) As above with removal of sodium polyacrylate beads by desiccation, including convection, conduction and radiation
15.) Incorporation of gas or fluid-filled devices or containment sacks of a desired shape and size within an organic substrate with a mycelial matrix binder that are removable through draining of the sack fluid and subsequent, non-disruptive removal of the sack from the grown matrix.
   For example, this process includes placing an inflated balloon within a mass of mycelial matrix, allowing for mycelial growth completion, and removing the balloon by popping, leaving behind a cavity the size and shape of the inflated balloon.
16.) Incorporation of gas or fluid-filled devices or containment sacks of a desired shape and size within a liquid mycelial inoculum that are removable through draining of the sack fluid and subsequent, non-disruptive removal of the sack from the grown matrix.
17.) Incorporation of solid shapes or structures that are later removed without damage to the resultant matrix and have in some way created functional pathways, flow paths or other utility passages, as in a lost cast method.
   For example, this process includes inclusion of shaped wax forms within the mycelial matrix that can be melted and drained through a small intentional hole and which leaves behind a cavity in the size and shape of the original wax form.
18.) Incorporation of a removable body that, when removed, leaves void spaces that are later refilled with additional cellular matrix by means of cellular regrowth, or is refilled with an alternate material not derived from the original cellular matrix.
   For example, this process includes inclusion of shaped wax forms within the mycelial matrix that can be melted and drained through a small intentional hole and which leaves behind a cavity in the size and shape of the original wax form and then allowing additional cellular growth of the pure mycelial structure into the cavity such that the cavity in the shape of the original wax shape is now a purely mycelial structure in that shape.
19.) Incorporation of a body of puffed grain that is removable through mycelial ingestion with subsequent replacement by a mycelial matrix.
   For example, this process requires mycelial colonization of a non-digestible or partially digestible filler material in which digestible fragments are incorporated that are later fully digested by mycelium, in that the digestion of the fragments necessitates suffusion of the mycelium onto the particle and in that when the particle is digested, only mycelium is left in the resultant space.

### Applications

The method of the invention may be utilized for any application in which the desired resultant product is a low-density, hydrophobic, high-porosity myceliated material with physical qualities attributed to some foams, but is biodegradable and non-toxic to produce, handle and dispose of, and where the production of such a product is not innately produced through standard processes. Some specific applications include but are not limited to: allocation of the material as an insulation mat; discrete and highly-specific allocation of the material as a biological tissue substitute or scaffold; substitution of non-biodegradable and/or petroleum based foams with the essentially inert material. Additional applications may be made in the medical, construction, and entertainment industries, as well as for artistic and personal ventures.

Additionally, due to the nature of the growth of the mycelial matrix and the customizable addition of void space fillers and substrates, products with variable density can be created including products that can contain millimeter (mm) scale and inch scale components coexisting within the same mycleial matrix.

For example, a highly porous, low density mycelial matrix can be grown in concert with a low porosity, high density mycelial matrix to produce two discrete product qualities within the same unit of production. Specifically, a replica of the human heart with foam-like, compressive ventricles and firm chamber walls could potentially be grown concurrently. Or, within a separate context, a very soft seat cushion for posterior support could be fashioned in concert with a firmer surrounding seat area as one discrete unit.

Variable density allocation could also be utilized to produce upright standing panels in which the bottom is heavier and denser, and the weight and density of the singular panel decreases from bottom to top. The resultant product could provide a supportive, rigid foundation at the base and a more flexible, insulating panel at the top.

Also, this process may be utilized for any application in which the desired resultant product is a formed, solely mycelial matrix where the initial form or shape is in some way inflated as a scaffold to support mycelial growth and which is then removed without disturbing resultant growth when the requisite time period concludes. Specifically, a latex-based form, which when inflated results in the shape of a human organ and which serves as a scaffold for mycelial growth and then is removed after deflation, leaving behind a reproduction of the organ.

The method of the invention may be characterized with the following steps;
A0. Make or procure void space fillers
A1. Make inoculum for pure mycelial matrix
A2. Add inoculum to void space fillers in predetermined shape or geometry
A3. Allow mycelial colonization to complete matrix formation
A4. Treat matrix to remove void space fillers
A5. Apply post-treatment to remaining mycelial matrix
-or-
B0. Inoculate substrate for composite matrix and allow partial mycelial colonization
B1. Disassemble colonized matrix and introduce predetermined percentage of pore space fillers
B3. Allow additional growth as need to complete matrix formation
B4. Treat matrix to remove void space fillers
B5. Apply post-treatment to remaining mycelial composite matrix.

The invention thus provides a mycelium foam with macroscopic void spaces as well as methods for making a pure mycelium foam with macroscopic void spaces or a composite mycelium foam with macroscopic void spaces.

The invention also provides relatively simple techniques for producing mycelial foams with macroscopic voids.

What is claimed is:

1. A mycelium foam comprising
a body of pure mycelium, and
at least one macroscopic void space within said body.

2. A mycelium foam as set forth in claim 1 comprising a plurality of macroscopic void spaces within said body.

3. A mycelium foam as set forth in claim 2 wherein said void spaces constitute at least 10% of said body.

4. A mycelium foam as set forth in claim 2 wherein said void spaces are sized from 0.04 inch to 3 inch.

5. A mycelium foam as set forth in claim 2 wherein said void spaces are discrete within said body.

6. A mycelium foam as set forth in claim 2 further comprising an additive in at least some of said void spaces.

7. A mycelium foam as set forth in claim 6 wherein said additive is a mineral solid.

8. A self-assembled fungal cellular matrix comprising
a composite matrix of non-nutrient discrete particles and mycelium, and
a plurality of macroscopic void spaces within said matrix.

9. A method of making a mycelium foam comprising the steps of
providing a tool defining a cavity therein;
packing said cavity of the tool with a plurality of spaced apart filler elements;
adding a fungus to said filler elements; and
allowing said fungus to grow mycelium within said cavity to form a pure mycelial matrix and to allow the mycelium to individually encapsulate said filler elements with said matrix.

10. A method as set forth in claim 9 further comprising the step of removing said filler elements from said matrix to produce a fungal cellular matrix having a plurality of macroscopic void spaces therein.

11. A method as set forth in claim 9 wherein said filler elements are agar beads.

12. A method as set forth in claim 9 wherein said filler elements are sodium polyacrylate beads.

13. A method as set forth in claim 9 wherein said filler elements are beeswax pellets.

14. A method of making a mycelium foam comprising the steps of
providing a tool defining a cavity therein;
packing said cavity of the tool with a substrate comprised of non-nutrient discrete particles and nutrient particles;
adding a fungus to said substrate;
allowing said fungus to grow mycelium within said substrate;
thereafter adding a plurality of spaced apart filler elements to said substrate;
allowing said fungus to grow mycelium within said cavity to form a composite matrix of said non-nutrient discrete particles and mycelium and to allow said mycelium to individually encapsulate said filler elements with said composite matrix.

15. A method as set forth in claim 14 further comprising the step of removing said filler elements from said composite matrix to produce a composite matrix having a plurality of macroscopic void spaces therein.

16. A method as set forth in claim 15 wherein said filler elements are agar beads.

17. A method as set forth in claim 15 wherein said filler elements are sodium polyacrylate beads.

18. A method as set forth in claim 15 wherein said filler elements are beeswax pellets.

19. A method of making a mycelium foam comprising the steps of
providing an organic substrate with a mycelial matrix binder;
adding a plurality of spaced apart filler elements to said substrate; and
allowing said fungus to grow mycelium within said substrate to form a composite matrix of said substrate and mycelium and to allow said mycelium to individually encapsulate said filler elements with said composite matrix.

20. A method as set forth in claim 19 further comprising the step of removing said filler elements from said composite matrix to produce a composite matrix having a plurality of macroscopic void spaces therein.

21. A method as set forth in claim 19 wherein said organic substrate is composed of aspen chips approximately 1×1×1 mm in size and said filler elements are agar beads approximately 2.5 mm in diameter.

22. A method as set forth in claim 21 wherein said aspen chips are in a ratio to said agar beads of 70 dry grams of aspen chips to 60 grams of agar beads.

23. A method as set forth in claim 19 wherein said organic substrate is composed of aspen chips approximately 1×1×1 mm in size and said filler elements are hydrated sodium polyacrylate beads.

24. A method as set forth in claim 23 wherein said aspen chips are in a ratio to said agar beads of 70 dry grams of aspen chips to 250 grams of sodium polyacrylate beads.

25. A method as set forth in claim 13 wherein said organic substrate is composed of aspen chips approximately 1×1×1 mm in size and said filler elements are beeswax pellets approximately 1.0 mm in diameter.

26. A method as set forth in claim 19 wherein said filler elements are gas or fluid-filled devices or containment sacks.
